(19) United States
(12) Patent Application Publication
(10) Pub. No.: US 2003/0120191 A1
(43) Pub. Date: Jun. 26, 2003

(54) Protective enclosure for body support

(76) Inventor: Margarita Clement, Northridge, CA (US)

Correspondence Address: MARGARITA CLEMENT, 17537 BLYTHE STREET, NORTHRIDGE, CA, 91325, US

(21) Appl. No.: 10/329,648

(22) Filed: Dec. 26, 2002

Related U.S. Application Data
(63) Continuation-in-part of application No. 09/063,985, filed on Apr. 21, 1998, now Pat. No. 6,443,920.

(51) Int. Cl. A61F 13/00

(52) U.S. Cl. 602/23; 602/61

(57) ABSTRACT

The device is a soft, flexible human body enclosure designed to aid, comfort, protect and prevent Decubitis (bed sores) and skin breakdowns on bedridden or wheelchair bound patients. The advantage of this device is that it covers major bony prominences, e.g. hips, lower back, buttocks, coccyx area and other pressure points from hips to knees. This device is also intended to promote healing of existing inflammations by holding dressings in place.

The protective enclosure for body support (10) comprises abdomen region support wrap (15) and two individual legs support wraps (25) and (27) sewn together at two specific places (45) to create one item, the protective enclosure for body support (10). The outer lining is made of sturdy material as nylon, rayon, acrylic, and cotton, Dacron, polyester or any other suitable material to make fabric. Inner filler is made of soft and pliable composite material, such as polyester fiber, acrylic fiber, foam rubber, enveloped by the outer lining of support wraps (15), (25) and (27). Velcro latches (30) and (40) at predetermined locations provide means for attachment when the support is closed around the abdomen region and the legs. For an additional comfort one or more detachable pads (20) of the same material as specified for body wraps lining and filler of various sizes and shapes can be added to the device.

For sanitary reasons, the whole body support enclosure (10) and the detachable pads (20) can be made disposable by substituting the external fabric of support wraps (15), (25) and (27) with suitable disposable material.

predetermined configuration and prevent irritation to bed sores caused by movement of the patient for purposes of comfort or administering personal hygiene.

### SUMMARY OF THE INVENTION

Empirical studies and prior literature provide sufficient evidence to conclude that Decubitis, bed sores, and inflamed tissue pose serious problems to persons confined to bed or wheelchair for prolonged periods or whose incapacitation prevent voluntary movements.

The problem arises from (a) contact with firm bed mattresses or wheelchair seats and (b) infrequent rotation of the patient for long periods. In most cases, the breakdown of skin occurs in the area of bony prominences at the hips, buttocks and knees where there is insufficient subcutaneous tissue, and, to a lesser extent, the upper back or shoulders where the body makes contact with the bed or other devices to rest on.

While physicians order topical medication and pads for the affected areas, these dressings cover only those inflamed areas while failing to prevent similar ulcerated areas elsewhere. The protective enclosing body support of the invention will protect extremities from the abdomen region to knees vulnerable to Decubitis.

The design of my invention allows for the placement of the individual on the device while it is open and in flat configuration. The support is than simply closed with adjustable Velcro latches, which facilitates proper fit and comfort.

The device is also designed with an opening to provide for urination or secretion without removing the device from the patient. For purpose of bathing or personal hygiene, the Velcro latches are released and the enclosure is opened to allow free access to the patient without discomfort.

The objects and advantages of the flexible support wrap of the present invention are:

(a) to relief pain and discomfort from any existing inflammation, or remove pressure to the skin from bony prominences, with particular attention to the abdomen region, comprising the lumbar area, the buttock, the hips, coccyx, and other pressure points from hips to knees;

(b) to keep the affected areas warm, thus stimulating blood circulation for bedridden or wheelchair bound patients unable to move frequently;

\mathring{c} to keep the skin protected from abrasive materials such as starched sheets and mattress covers and the residual laundry chemicals left on the sheets or garment, etc.;

(d) that the patient can attach or remove the enclosure with ease by himself/herself without assistance;

(e) that the device allows for movement of patient (rotation in bed) using the device and not the patient;

(f) that the patient may also move to and from bed or chair with minimal discomfort not having to reposition or re-fit the device;

(g) that the device permits ease of access through opening to areas requiring attention to personal hygiene, urination and secretion, without removing the device.

Further objects and advantages are to provide that the protective enclosing device is easily and conveniently opened and closed with minimal discomfort to the patient, which can increase muscular, skin and vascular tone through unrestricted blood circulation. Among other advantages, the present invention eliminates the need for rubber rings or doughnuts that merely increase the pressure around bony prominences. The present device protects major bony prominences from abdomen region to knees in contact with mattress or chair simultaneously, yet allows the patient to freely move (rotate or sit) with the device attached to the body. This device does not discriminate any patient based on sex or age, and any patient or person with potential susceptibility for skin breakdown can use it.

In brief, the present invention provides a body support enclosure to prevent and protect bedridden patients from bedsores and skin breakdown, and to provide relief from pain and discomfort from existing inflammation.

### BRIEF DESCRIPTION OF THE DRAWINGS

The present invention will be better understood by reference to the drawings wherein:

FIG. 1 is a plan view of the protective enclosure for body support 10 according to the present invention while open, comprising abdomen region support wrap 15 and legs support wraps 25 and 27 sewn together at two specific places 45. It is also showing the detachable extra pads 20 for an additional comfort, placed as needed at affected areas.

FIG. 2 is a perspective view showing the protective enclosure for body support 10 as it would appear when wrapped around a patient's body, cushioning bony prominences of the abdomen region, hips, thighs and knees.

### DETAILED DESCRIPTION

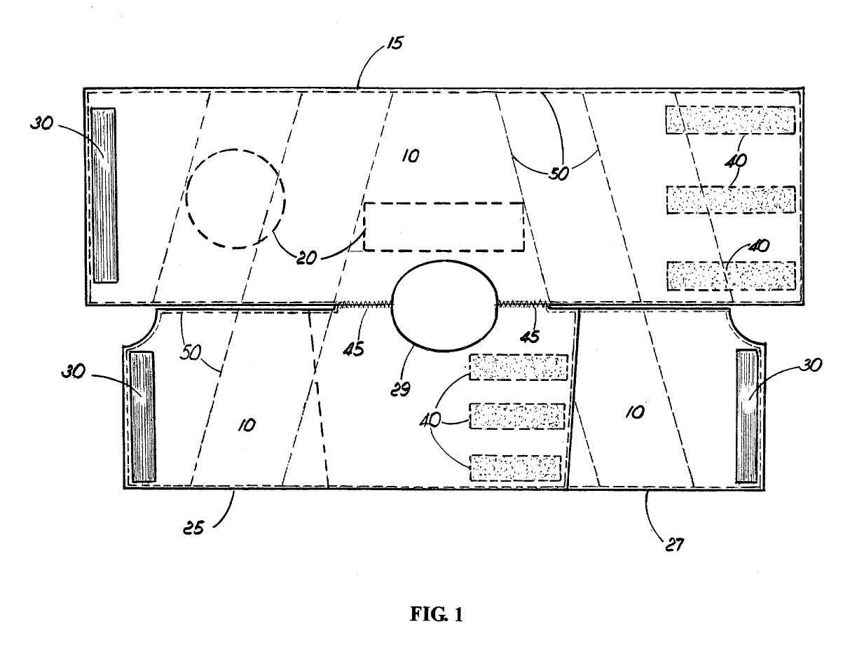

A preferred embodiment of the present invention is illustrated in FIG. 1. The protective enclosure for body support device 10 comprises three parts sewn together at two specifics places 45. The abdomen region support wrap of rectangular shape 15 and two legs support wraps of rectangular shape 25 and 27 overlapping each other as shown in the FIG. 1. The outer lining is made of sturdy material as nylon, rayon, cotton, Dacron, polyester, various blends or any other material to make fabrics.

The filler material is made of soft and pliable composite material such as polyester fiber, acrylic fiber, foam rubber (flat sheet, egg-shaped, aerated, etc.) or any other soft cushioning material, providing an effect of preventing contact between the patient's bony prominences and bed or chair, and to allow inflamed areas of the patient body to breathe and benefit from increase of blood circulation to existing skin lesions or bed sores.

The wraps are sewn around the perimeter and several other places with thread 50 of suitable material compatible to the materials of outer lining.

For an additional comfort one or more detachable pads 20 of various sizes and shapes (round, rectangular, oblong, etc.) could be added to the device at locations of the body where extra padding is needed to further cushion bony prominences of the patient. The pads will be manufactured from the same specified materials as for body wraps lining and filler. The pads shall be attached to support wraps on the inside by means of peel-off both sided adhesive tape or by any other suitable means.

For sanitary reasons, the external fabric of support wraps 15, 25 and 27, and detachable pads 20 can be made also from disposable suitable material in order to make the detachable pads or the whole body support enclosure disposable.

All material used are non-allergenic.

The rectangular abdomen region support wrap 15 and the legs support wraps 25 and 27 can be made also with rounded or beveled corners to facilitate ease of manufacturing.

For the purpose of administering personal hygiene (urination or secretion), an opening 29, as cutouts in the wraps, is provided.

Velcro latches or fasteners 30 and 40 are sewn to the support wraps to facilitates opening and closing of the device as well as allow for adjustment to body size and patient's comfort.

The Velcro latch or fastener comprises a hook tape 30 and a loop tape 40. The loop tapes 40 are made longer to permit adjustability and thereby fit the support wrap to the correct size for the patient. The number of Velcro loop tapes 30 will vary according to the size of the enclosure device.

A set of loop handles made out of sturdy fabric can be attached to the outer side of abdomen and legs wraps, two at each side, to facilitate rotation of bedridden patients.

A size of present invention will vary with the size of the patient: small, medium and large, which also cover variation in accordance with sex, age and body measurements.

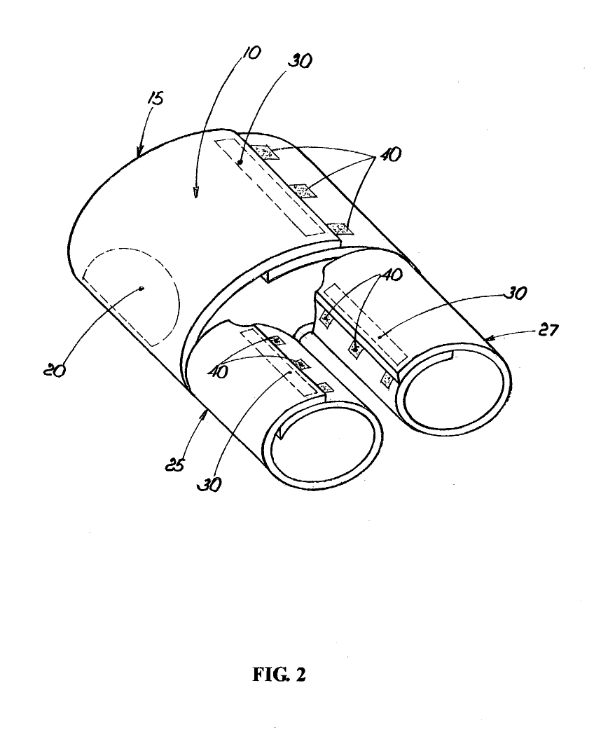

A perspective view of the present invention is shown in FIG. 2, illustrating the enclosure body support as it would appear when closed around a patient's body by Velcro fasteners.

Further advantages of the protective enclosure for body support wrap 10 include:

(a) a bedridden or wheelchair bound patient will be relieved of pain and discomfort caused by hard contact with mattresses and seats;

(b) the cushioning effect will prevent development of inflamed tissue around bony prominences and allow the curative effect of increased blood circulation to existing lesions or bed sores (Decubitis);

\mathring{c} the Velcro fasteners will permit quick and easy opening and closing of the device for purpose of bathing, changing undergarments, personal hygiene, and application of topical medication or new dressings.

The manner of using the protective enclosure and body support is simple and convenient, either for the attending health care provider, family member or for the patient himself/herself. The patient is places with the back on the opened device so that the abdomen region of the patient rest on the abdomen wrap 15. The patient is than centered on the abdomen wrap 15 with the buttocks coccyx area slightly above the opening 29 and with legs overlying the legs wraps 25 and 27. The protective enclosure for body support wrap 10 is then closed covering the body from the abdomen region to the knees, protecting and cushioning the bony prominences of hips, buttocks, upper parts of the legs and knees and at the same time a cutout 29 permits urination and secretion.

When the wrap is closed, the device is held in place on the patient's body by the Velcro latches 30 and 40, adjusted for body size and patient's comfort. Opening of the device is equally simple and easy by pulling the Velcro hook tape 30 and loop tape 40 apart to free the overlapping edges of the support.

The present invention thus protects and supports the body easily and conveniently, can be closed and reopened as often as needed as treatment or hygiene requires, and can be removed without discomfort to the individual. The invention thus relieves pressure on skin and subcutaneous tissue; prevents sensory loss, or absence of the patient's awareness of pain and pressure; permit's the supply of nutrients to tissue cells, thus avoiding edema, by improving blood circulation which, in turn, aids the healing of existing ulcers or bed sores; and facilitates increased activity by the patient because the device reduces or eliminates the discomfort of motion, allowing activity to thereby increase muscular, skin, and vascular tone.

Although the description above refers to certain specifics, these should not be construed as limiting the scope of the invention. The protective body support will also prevent exacerbation of dermatological anomalies to bacterial and viral infection, such as HIV/AIDS.

What I claim is:

1. A soft, flexible, human protective enclosure for body support to comfort, protect, and prevent bed sores (Decubitis) and skin breakdowns on bedridden, or wheelchair bound patients or persons, by cushioning bony prominences of hips, lower back, buttock, coccyx area, and other pressure points from abdomen region to knees, comprising:
(a) an abdomen region support wrap of rectangular shape and two individual slightly irregular rectangular legs support wraps sewn together at two specific places to create one item, the protective enclosure for body support;
(b) a body support according to claim 1(a) wherein said rectangular shape support wraps corners are rounded or beveled to facilitate manufacturing;
\mathring{c} the wraps are sewn around perimeters, and several other places for added strength, with thread of suitable material as nylon, polyester, acrylic, cotton or other suitable material compatible to the material of outer lining;
(d) a one or more detachable pads of various sizes and shapes, round, rectangular, oblong etc. of the same specified material as for support wraps for added cushioning to affected areas, attached to inside of support wraps by means of both sided adhesive peel-off tape or by any other suitable means;
(e) a Velcro latches, or fasteners of predetermined size, sewn to the support wraps at predetermined locations comprising of hook and loop tape, whereas the loop tapes are made longer to permit adjustability to patient's body size;
(f) a number of Velcro loop tapes or fasteners of predetermined length located parallel each other for larger support wrap sizes or of one piece for smaller sizes;
(g) a set of loop handles made of sturdy fabric material attached to the outer side of abdomen and legs wraps, two at each side, to facilitate movement or rotation of bedridden patients;
(h) an opening, cutout of round, square or oblong shape, in the wraps for the purpose of administering personal hygiene, urination and secretion.

2. A body support according to the claim 1 wherein the outer lining of abdomen region support wrap and of two legs support wraps are made of sturdy material as nylon, rayon, acrylic, cotton, Dacron, polyester, various blends or any other suitable materials to make fabric.

3. A body support according to claim 1 wherein the filler material is made of soft and pliable composite material, such as polyester fiber, acrylic fiber or other fibrous materials of, predetermined specific density to provide cushioning effect.

4. A body support according to claim 3 wherein the filler material is made of soft and pliable composite material, such as foam rubber flat sheet, egg-shaped or aerated of predetermined specific density.

5. A size of the protective body enclosure support will vary with the size of the patient and shall be made at least in three different sizes: small, medium and large to accommodate most sizes variances.

6. For sanitary reasons, the whole body support enclosure and the detachable pads can be made disposable by substituting the external fabric of support wraps with suitable disposable material.
